(19) United States
(12) Patent Application Publication
(10) Pub. No.: US 2024/0390500 A1
(43) Pub. Date: Nov. 28, 2024

(54) PHARMACEUTICAL COMPOSITION COMPRISING A GLP-1 AGONIST, AN INSULIN AND METHIONINE

(71) Applicant: Sanofi-Aventis Deutschland GmbH, Frankfurt (DE)

(72) Inventors: Annika Hagendorf, Frankfurt am Main (DE); Gerrit Hauck, Frankfurt am Main (DE); Werner Mueller, Frankfurt am Main (DE); Isabell Schoettle, Frankfurt am Main (DE); Verena Siefke-Henzler, Frankfurt am Main (DE); Katrin Tertsch, Frankfurt am Main (DE)

(21) Appl. No.: 18/647,078

(22) Filed: Apr. 26, 2024

Related U.S. Application Data
(63) Continuation of application No. 17/366,332, filed on Jul. 2, 2021, now abandoned.
Application No. 17/366,332 is a continuation of application No. 16/666,000, filed on Oct. 28, 2019, now abandoned.
Application No. 16/666,000 is a continuation of application No. 15/803,589, filed on Nov. 3, 2017, now abandoned.
Application No. 15/803,589 is a division of application No. 13/509,542, filed on Aug. 2, 2012, now Pat. No. 10,029,011.

(30) Foreign Application Priority Data
Nov. 13, 2009 (DE) 102009052831.8
May 18, 2010 (DE) 102010020902.3

(51) Int. Cl. A61K 47/10; A61K 9/00; A61K 38/26; A61K 38/28; A61K 47/18; A61K 47/20; C07K 14/605; C07K 14/62

(52) U.S. Cl. CPC A61K 47/10; A61K 9/0019; A61K 38/26; A61K 38/28; A61K 47/183; A61K 47/20; C07K 14/605; C07K 14/62

(57) ABSTRACT

A liquid composition comprising a GLP-1 agonist or/and a pharmacologically tolerable salt thereof, an insulin or/and a pharmacologically tolerable salt thereof, and, optionally, at least one pharmaceutically acceptable excipient, wherein the composition comprises methionine, as add-on therapy with metformin where appropriate.

### RELATED APPLICATIONS

This application is a continuation of U.S. patent application Ser. No. 17/366, 332, filed Jul. 2, 2021, which is a continuation of U.S. patent application Ser. No. 16/666, 000, filed Oct. 28, 2019, which is a continuation of U.S. patent application Ser. No. 15/803, 589, filed Nov. 3, 2017, which is a divisional application of U.S. patent application Ser. No. 13/509, 542, filed Aug. 2, 2012, now U.S. Pat. No. 10, 029, 011, which is a 35 U.S.C. § 371 filing of International Patent Application No. PCT/EP2010/067250, filed Nov. 11, 2010, which claims priority to German Patent Application Nos. 102009052831.8, filed Nov. 13, 2009, and 102010020902.3, filed May 18, 2010, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Apr. 22, 2024, is named 754103_081629-1253USDIVCON3_ST26.xml and is 6, 129 bytes in size.

### DESCRIPTION

The present application relates to a liquid composition comprising a GLP-1 agonist or/and a pharmacologically tolerable salt thereof, an insulin or/and a pharmacologically tolerable salt thereof, and, optionally, at least one pharmaceutically acceptable excipient, wherein the composition comprises methionine.

The present application further relates to the composition according to the present invention for treating diabetes mellitus. The present application further relates to the use of a composition according to the present invention in the manufacture of a pharmaceutical for treating diabetes mellitus. The present application further relates to a method for manufacturing a composition according to the present invention, comprising formulating a GLP-1 agonist or/and a pharmacologically tolerable salt thereof with an insulin or/and a pharmaceutically acceptable salt thereof, methionine, and, optionally, at least one pharmaceutically acceptable excipient. The present application further relates to a method for treating a patient with a composition according to the present invention, comprising administering the composition to the patient.

Customary compositions of insulin and GLP-1 compounds comprise an isotonicity agent, a buffer for adjusting the pH, and a preservative. A further frequently used constituent of insulin compositions is zinc, which forms a complex with insulin. This results in a delayed action of insulin being achieved.

WO 2003/020201 (Eli Lilly) relates to a liquid pre-mixed formulation comprising a GLP-1 compound and a basal insulin. A specific formulation contains Val⁸-GLP-1 or exendin-4, insulin NPH, glycerol as an isotonicity agent, m-cresol and phenol as preservatives, and a phosphate buffer at a pH of 7.6 or 8. Another formulation comprises exendin-4 or Val⁸-Glu²²GLP-1 (7-37) OH, insulin glargine (Lantus), zinc, glycerol and mannitol as isotonicity agents, m-cresol as a preservative, zinc, and sodium acetate, pH 4.

WO 2004/035623 (Zealand Pharmaceuticals) discloses a liquid composition comprising a stabilized exendin, 50 mM histidine, 100 to 200 mM sucrose, mannitol or other acceptable sugar, 20 mM methionine, 20 mM asparagine-glutamine or Asp, at a pH of 5.3. Stabilization is effected by certain modifications of the amino acid building blocks of exendin-4 (1-39), for example, at positions Gln13, Met14, Trp25, or Asn28. This composition does not comprise insulin.

WO 2005/046716 (Novo Nordisk) discloses liquid compositions which comprise liraglutide and insulin aspart, a buffer with a pH of 7.7, poloxamer 188 as a surfactant, phenol, propylene glycol, and, optionally, zinc. Without poloxamer 188, the compositions were unstable. With polysorbate 20, stabilization was achieved.

WO 2006/029634 (Novo Nordisk) relates to liquid pharmaceutical compositions which comprise an insulinotropic peptide (GLP-1 agonist), an insulin peptide, and a ligand for His^{B10} (ligand of His at position 10 of the B chain of insulin). The composition can comprise polysorbate-20 or poloxamer 188 as a surfactant. Specific compositions disclosed in this document comprise human insulin or human B28 Asp insulin (insulin aspart), liraglutide (GLP-1 agonist), glycerol as an isotonicity agent, zinc acetate, pH 7.4 or 7.9. Depending on the amount of insulin used or of liraglutide, these compositions were, in part, already unstable after 15 days of storage at room temperature. Stability of these compositions was achieved by adding a ligand for His^{B10}. Further formulations consisted of liraglutide, insulin aspart or detemir, propylene glycol, phenol, and phosphate buffer, pH 7.7. These compositions were practically immediately unstable. Adding poloxamer-188 or polysorbate-20 and a ligand for His^{B10} led to stabilization.

WO 2006/051103 (Novo Nordisk) discloses liquid compositions which comprise detemir (a basal insulin), liraglutide (GLP-1 compound), and poloxamer 188 or polysorbate 20 as a surfactant. Further constituents are phenol, NaCl, propylene glycol, zinc acetate, and sodium phosphate buffer or glycylglycine buffer (pH 7.7). m-Cresol is present in some of these compositions. By adding poloxamer 188 or polysorbate 20, the compositions could be stabilized.

WO 2008/124522 (Biodel) relates to compositions which comprise an insulin, a zinc chelator (e.g., EDTA or EGTA), and a GLP-1 analog.

About 120 million people around the world suffer from diabetes mellitus. These include about 12 million type I diabetics, for whom replacement of the deficient endocrine insulin secretion is the only possible therapy at present. Those affected are dependent on insulin injections for life, usually several times a day. Type II diabetes contrasts with type I diabetes in that there is not always a deficiency of insulin, but in a large number of cases, especially at the advanced stage, treatment with insulin, where appropriate in combination with an oral antidiabetic, is considered the most advantageous form of therapy.

In healthy individuals, release of insulin by the pancreas is strictly coupled to the blood glucose concentration. Elevated blood glucose levels, like those occurring after meals, are quickly compensated by a corresponding rise in insulin secretion. In the fasting state, the plasma insulin level falls to a basal value which is sufficient to ensure a continuous supply of glucose to insulin-sensitive organs and tissues, and to keep hepatic glucose production low in the night. The replacement of the endogenous insulin secretion by exogenous, usually subcutaneous administration of insulin does not in general come close to the above-described quality of the physiological regulation of blood glucose. Frequently there are instances of blood glucose being thrown off-track, either upwardly or downwardly, and in their most severe forms these instances may be life-threatening. In addition, however, blood glucose levels which are elevated over years, without initial symptoms, constitute a considerable health risk. The large-scale DCCT study in the USA (The Diabetes Control and Complications Trial Research Group (1993), N. Engl. J. Med. 329, 977-986) showed unambiguously that chronically elevated blood glucose levels are responsible for the development of late diabetic complications. Late diabetic complications are microvascular and macrovascular damage which is manifested in certain circumstances as retinopathy, nephropathy, or neuropathy, and leads to blindness, renal failure, and loss of extremities, and, in addition, is associated with an increased risk of cardiovascular disorders. From this it can be inferred that an improved therapy of diabetes must be aimed primarily at keeping blood glucose as closely as possible within the physiological range. According to the concept of intensified insulin therapy, this is to be achieved by means of injections, several times a day, of fast-acting and slow-acting insulin preparations. Fast-acting formulations are given at meal times, in order to compensate the postprandial rise in blood glucose. Slow-acting basal insulins are intended to ensure the basic supply of insulin, especially during the night, without leading to hypoglycemia.

Insulin is a polypeptide composed of 51 amino acids which are divided between two amino acid chains: the A chain, with 21 amino acids, and the B chain, with 30 amino acids. The chains are linked together by 2 disulfide bridges. Insulin preparations have been employed for many years in diabetes therapy. Such preparations use not only naturally occurring insulins but also, more recently, insulin derivatives and insulin analogs.

Insulin analogs are analogs of naturally occurring insulins, namely human insulin or animal insulins, which differ by replacement of at least one naturally occurring amino acid residue by other amino acids and/or by addition/deletion of at least one amino acid residue, from the corresponding, otherwise identical, naturally occurring insulin. The amino acids in question may also be amino acids which do not occur naturally.

Insulin derivatives are derivatives of naturally occurring insulin or of an insulin analog which are obtained by chemical modification. The chemical modification may consist, for example, in the addition of one or more defined chemical groups onto one or more amino acids. Generally speaking, the activity of insulin derivatives and insulin analogs is somewhat altered as compared with human insulin.

Insulin analogs with an accelerated onset of action are described in EP 0 214 826, EP 0 375 437, and EP 0 678 522. EP 0 124 826 relates, among other things, to replacements of B27 and B28. EP 0 678 522 describes insulin analogs which have different amino acids in position B29, preferably proline, but not glutamic acid. EP 0 375 437 encompasses insulin analogs with lysine or arginine at B28, which may optionally also be modified at B3 and/or A21.

EP 0 419 504 discloses insulin analogs which are protected from chemical modifications by modification of asparagine in B3 and of at least one further amino acid at positions A5, A15, A18 or A21.

WO 92/00321 describes insulin analogs in which at least one amino acid in positions B1-B6 has been replaced by lysine or arginine. Such insulins, according to WO 92/00321, have an extended effect. A delayed effect is also exhibited by the insulin analogs described in EP-A 0 368 187.

The commercially available preparations of naturally occurring insulins for insulin replacement differ in the origin of the insulin (e.g., bovine, porcine, human insulin) and also in their composition, and thereby the activity profile (onset and duration of action) may be influenced. Through combination of different insulin products it is possible to obtain any of a very wide variety of activity profiles and to bring about very largely physiological blood sugar values. Recombinant DNA technology nowadays allows the preparation of modified insulins of this kind. They include insulin glargine (Gly (A21)-Arg (B31)-Arg (B32) human insulin, Lantus), with an extended duration of action. Insulin glargine is injected in the form of a clear, acidic solution, and owing to its dissolution properties is precipitated, in the physiological pH range of the subcutaneous tissue, as a stable hexamer association. Insulin glargine is injected once a day and is notable in comparison with other long-active insulins for its flat serum profile and the associated reduction in the risk of night hypoglycemias (Schubert-Zsilavecz et al., 2:125-130 (2001)).

The specific preparation of insulin glargine that leads to the prolonged duration of action is characterized by a clear solution with an acidic pH.

Exendins are a group of peptides which can lower blood glucose concentrations. Exendins have a certain similarity to the sequence of GLP-1 (7-36) (53%, Goke et al. J. Biol Chem 268, 19650-55). Exendin-3 and exendin-4 stimulate an increase in cellular cAMP production in the acinar cells of the guinea pig pancreas by interacting with exendin receptors (Raufman, 1996, Reg. Peptides 61:1-18). Exendin-3, in contrast to exendin-4, effects an increase in the release of amylase in the acinar cells of the pancreas. Exendins act as GLP-1 agonists.

Glucagon-like peptide 1 (GLP-1) is an endocrine hormone which enhances the insulin response following oral intake of glucose or fat. In general, GLP-1 lowers glucagon concentrations, slows gastric emptying, stimulates (pro) insulin synthesis, enhances sensitivity to insulin, and stimulates insulin-independent glycogen synthesis (Holst (1999), Curr. Med. Chem 6:1005, Nauck et al. (1997) Exp Clin Endocrinol Diabetes 105:187, Lopez-Delgado et al. (1998) Endocrinology 139:2811). Human GLP-1 has 37 amino acid residues (Heinrich et al., Endocrinol. 115:2176(1984), Uttenthal et al., J Clin Endocrinol Metabol (1985) 61:472). Active fragments of GLP-1 include GLP-1 (7-36) and GLP-1 (7-37).

Exendin-3, exendin-4 and exendin agonists have been proposed for treating diabetes mellitus and preventing hyperglycemia, by reducing gastric motility and gastric emptying (U.S. Pat. No. 5, 424, 286 and WO98/05351).

Exendin analogs can be characterized by amino acid substitutions and/or C-terminal truncation of the native exendin-4 sequence. Such exendin analogs are described in WO 99/07404, WO 99/25727, and WO 99/25728.

Solid-phase synthesis of AVE0010 is described in WO 01/04156 A1. AVE0010 has the sequence: desPro³⁶dexendin-4 (1-39)-Lys₆-NH₂. This substance is published as SEQ ID NO:93 in WO 01/04156:

(SEQ ID NO: 1)
H-G-E-G-T-F-T-S-D-L-S-K-Q-M-E-E-E-A-V-R-L-F-I-E-W-
L-K-N-G-G-P-S-S-G-A-P-P-S-K-K-K-K-K-K-NH₂

Exendin-4 (39 AS) has the sequence:

(SEQ ID NO: 2)
H-G-E-G-T-F-T-S-D-L-S-K-Q-M-E-E-E-A-V-R-L-F-I-E-W-
L-K-N-G-G-P-S-S-G-A-P-P-P-S-NH₂

Exendin-3 has the sequence (J. Bio. Chem., 267, 1992, 7402-7405):

(SEQ ID NO: 3)
H-His-Ser-Asp-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-NH₂

GLP-1 has the sequence:

(SEQ ID NO: 4)
H-A-E-G-T-F-T-S-D-V-S-S-Y-L-E-G-Q-A-A-K-E-F-I-A-W-
L-V-K-G-R-NH₂

It is an object of the present invention to increase the stability of liquid formulations comprising a GLP-1 agonist and an insulin. More particularly, it is an object of the present invention to improve physical and chemical integrity. We have found that this object is achieved by formulating the GLP-1 agonist and the insulin with methionine.

It was found that methionine is able to increase the storage stability of a composition comprising a GLP-1 agonist such as AVE0010 and an insulin such as insulin glargine. Methionine does not affect the physical integrity of these compositions.

The stability of pharmaceutically active polypeptides can be impaired by various mechanisms. These include pH, temperature, light, and the effects of certain constituents.

In connection with the present invention, it was found that a range of customary constituents of insulin formulations or of formulations of GLP-1 agonists are disadvantageous for the chemical or/and physical integrity and the storage stability of formulations which comprise an insulin and a GLP-1 agonist. These are, for example, acetate, polysorbate 20, polysorbate 80, poloxamer 188, benzalkonium chloride, and lysine. The compositions according to the present invention are therefore preferably free of these constituents.

The present invention accordingly provides for a liquid composition comprising a GLP-1 agonist or/and a pharmacologically tolerable salt thereof, an insulin or/and a pharmacologically tolerable salt thereof, and, optionally, at least one pharmaceutically acceptable excipient, wherein the composition comprises methionine.

The composition according to the present invention preferably comprises methionine in an amount ranging from 0.5 mg/mL to 20 mg/mL, more preferably in an amount ranging from 1 mg/mL to 5 mg/mL, especially preferably in an amount of 3.0 mg/mL. Methionine in the D-form can be used. Likewise, methionine in the L-form can be used. Likewise, mixtures of the D-form and the L-form in any desired proportions can be used.

More particularly, the composition according to the present invention is free of surfactants, such as polyols and partial and fatty acid esters and ethers of polyhydric alcohols such as those of glycerol and sorbitol. The compositions according to the present invention are more particularly free of partial and fatty acid esters and ethers of glycerol and sorbitol selected from the group consisting of Span®, Tween®, Myrj®, Brij®, Cremophor®. Furthermore, the compositions according to the present invention are more particularly free of polyols selected from the group consisting of polypropylene glycols, polyethylene glycols, poloxamers, Pluronics, Tetronics. More particularly, the composition according to the present invention is free of at least one substance selected from the group consisting of polysorbate, polysorbate and poloxamer.

More particularly, the composition according to the present invention is substantially free, preferably free, of polysorbate, such as, for example, polysorbate 20.

More particularly, the composition according to the present invention is substantially free, preferably free, of polysorbate 80.

More particularly, the composition according to the present invention is substantially free, preferably free, of poloxamer, such as, for example, poloxamer 188.

More particularly, the composition according to the present invention is substantially free, preferably free, of benzalkonium chloride.

More particularly, the composition according to the present invention is substantially free, preferably free, of histidine.

More particularly, the composition according to the present invention is substantially free, preferably free, of EDTA, more particularly sodium EDTA.

More particularly, the composition according to the present invention is substantially free, preferably free, of histidine and sodium EDTA.

The composition according to the present invention can comprise one or more substances which are customarily used to buffer the pH (buffer substances). Examples of such buffer substances are acetate, citrate, and phosphate. More particularly, the composition according to the present invention can comprise one or more substances which are customarily used to buffer the pH in an amount which is sufficient, for example, as a counterion for the GLP-1 agonist or/and the insulin. The composition according to the present invention can comprise one or more buffer substances, for example, each in an amount of up to 1 mg/ml, up to 0.5 mg/ml, up to 0.1 mg/ml, up to 0.05 mg/ml, up to 0.02 mg/ml, or up to 0.01 mg/ml. The composition according to the present invention can likewise be substantially free of buffer substances. Preferably, the composition according to the present invention is free of buffer substances.

The composition according to the present invention can comprise acetate, for example, in an amount of up to 1 mg/ml, up to 0.5 mg/ml, up to 0.1 mg/ml, up to 0.05 mg/ml, up to 0.02 mg/ml, or up to 0.01 mg/ml. These amounts are, for example, sufficient as a counterion for the GLP-1 agonist. Likewise, the composition according to the present invention can be substantially free of acetate. Preferably, the composition according to the present invention is free of acetate.

The composition according to the present invention can comprise citrate, for example, in an amount of up to 1 mg/ml, up to 0.5 mg/ml, up to 0.1 mg/ml, up to 0.05 mg/ml, up to 0.02 mg/ml, or up to 0.01 mg/ml. These amounts are, for example, sufficient as a counterion for the GLP-1 agonist. Likewise, the composition according to the present invention can be substantially free of citrate. Preferably, the composition according to the present invention is free of citrate.

The composition according to the present invention can comprise phosphate, for example, in an amount of up to 1 mg/ml, up to 0.5 mg/ml, up to 0.1 mg/ml, up to 0.05 mg/ml, up to 0.02 mg/ml, or up to 0.01 mg/ml. These amounts are, for example, sufficient as a counterion for the GLP-1 agonist. Likewise, the composition according to the present invention can be substantially free of phosphate. Preferably, the composition according to the present invention is free of phosphate.

The pharmaceutical composition of the present invention can have an acidic or physiological pH. An acidic pH range is preferably in the range of pH 1-6.8, pH 3.5-6.8, or pH 3.5-5. A physiological pH is preferably in the range of pH 2.5-8.5, more preferably pH 4.0 to 8.5, even more preferably pH 6.0 to 8.5. Especially preferred is a pH of approximately 4.5. For pH adjustment, physiologically safe dilute acids (typically HCl) and alkalis (typically NaOH) are suitable.

The composition according to the present invention can comprise a suitable preservative. Suitable preservatives are, for example, phenol, m-cresol, benzyl alcohol, and/or p-hydroxybenzoate esters. m-Cresol is preferred. However, a preservative can also be omitted.

The composition according to the present invention can comprise zinc ions. The concentration of the zinc ions is preferably in the range from 1 μg/ml to 2 mg/ml, more preferably in the range from 5 μg to 200 μg zinc/ml, more particularly at a maximum of 0.06 mg/ml, especially preferably at 0.06 mg/ml.

Furthermore, the composition according to the present invention can comprise suitable isotonicity agents. Suitable isotonicity agents are, for example, glycerol, dextrose, lactose, sorbitol, mannitol, glucose, NaCl, calcium or magnesium compounds such as CaCl, etc. The concentrations of glycerol, dextrose, lactose, sorbitol, mannitol, and glucose are customarily in the range of 100-250 mM, NaCl in a concentration of up to 150 mM. Glycerol is preferred. More particularly, 85% glycerol at 20.0 mg/ml is preferred.

The composition according to the present invention can further comprise further additives, such as salts, which retard the release of at least one insulin. Preferably, the composition is free of these additives.

More particularly, the composition is intended for parenteral administration. The composition according to the present invention is preferably an injectable composition, more preferably for subcutaneous injection. More particularly, the composition of the present invention is suitable for injection once a day.

More particularly, the formulation according to the present invention has, after storage for 1 month, 2 months, 4 months, or 6 months at a temperature of +5° C. or 25° C., an activity of at least 80%, at least 90%, at least 95%, or at least 98% of the activity at the start of storage.

In the present application, “activity” can mean the activity of the insulin which is used in the formulation according to the present invention. Methods for determining the activity of insulin are known to a person skilled in the art.

In the present application, “activity” can likewise mean the activity of the GLP-1 agonist which is used in the formulation according to the present invention. Methods for determining the activity of a GLP-1 agonist are known to a person skilled in the art.

More particularly, the formulation according to the present invention exhibits chemical integrity after storage for 1 month, 2 months, 4 months, or 6 months. Chemical integrity means, more particularly, that after storage at a temperature of +5° C., 25° C., or 40° C. the formulation comprises at least 80%, at least 90%, at least 95%, or at least 98% of the active ingredient, compared with the start of storage, in a substantially chemically unchanged form.

Chemical integrity can mean the chemical integrity of the GLP-1 agonist. GLP-1agonists may comprise a methionine residue (e.g. position 14 in AVE0010). Chemical integrity of the GLP-1 agonist means, more particularly, that oxidation of the methionine residue is prevented.

Chemical intregrity can likewise mean the chemical integrity of the insulin.

Preferably, chemical integrity means the integrity of the insulin and the GLP-1 agonist.

More particularly, the formulation according to the present invention exhibits physical integrity after storage for 1 month, 2 months, 4 months, or 6 months. Physical integrity means, more particularly, that after storage at a temperature of +5° C., 25° C., or 40° C. the formulation comprises at least 80%, at least 90%, at least 95%, or at least 98% of the active ingredient, compared with the start of storage, in a substantially physically unchanged form.

Physical integrity can mean the integrity of the GLP-1 agonist. Likewise, physical integrity can mean the integrity of the insulin. Physical integrity means, more particularly, that the GLP-1 agonist or/and the insulin does/do not form aggregates, such as, for example, fibrils.

Preferably, physical integrity means the integrity of the insulin and the GLP-1 agonist.

The GLP-1 agonist is preferably selected from the group consisting of exendin-3 and analogs and derivates thereof, exendin-4 and analogs and derivates thereof, and in which case the GLP-1 agonist is more preferably selected from the group consisting of AVE0010 and exendin-4.

Exendin-3, analogs and derivates of exendin-3, exendin-4, and analogs and derivates of exendin-4 can be found in WO 01/04156, WO 98/30231, U.S. Pat. No. 5, 424, 286, EP application 99 610, 043.4, and WO 2004/005342. These documents are incorporated herein by reference. The exendin-3, exendin-4, and analogs and derivates thereof described in these documents can be synthesized by means of the methods described therein, after which modifications are optionally carried out.

The sequences of AVE0010 (SEQ ID NO:1), exendin-4 (SEQ ID NO:2), and exendin-3 (SEQ ID NO:3) show a high degree of similarity. The sequences of AVE0010 and exendin-4 are identical at positions 1-37. Sequence 1-39 from exendin-4 is at 37 of the 39 positions (94%) identical to the exendin-3 sequence at positions 48-86. With reference to the sequences, a person skilled in the art can readily convert the positions specified herein, which relate to a particular sequence (e.g. to the sequence of AVE0010 or exendin-4), to other sequences.

Analogs and derivates of exendin-3 or/and exendin-4 contain more particularly a modified amino acid sequence. For example, single amino acids can be deleted (e.g. desPro36, desPro37, desAsp28, desMet(O)14 in exendin-4 and the corresponding positions in exendin-3). Likewise, single positions can be substituted (e.g. Met(O)¹⁴, Trp(O₂)²⁵, IsoAsp²⁸, Asp²⁸ Pro³⁸ in exendin-4 and the corresponding positions in exendin-3), in which case unnatural amino acids such as Met(O) (methionine sulfoxide or methionine sulfone), Trp (O₂) (N-formylkynurenine), or/and IsoAsp (β-aspartate or isoaspartate) can also be used. Unnatural amino acids can be readily inserted, in the form of corresponding amino acid building blocks, into the sequence.

Furthermore, the C-terminus or/and the N-terminus can be modified, for example, by an additional sequence such as -(Lys)-, -(Lys)₂-, -(Lys)₃-, -(Lys)₄-, -(Lys)₅-, -(Lys)₆-, -Asn-(Glu)₅-, in which case -(Lys)₄-, -(Lys)₅-, -(Lys)₆-, -Asn-(Glu)₅- are preferred. The carboxyl group at the C-terminus is preferably modified to an amide group (—NH₂). Optionally, modification of the C-terminus or/and of the N-terminus is carried out as a further step after completion of synthesis.

Pharmaceutically tolerable salts can be manufactured in a further step after completion of the synthesis cycles of the method according to the present invention.

The manufacture of pharmaceutically tolerable salts of peptides is known to a person skilled in the art. A preferred pharmaceutically tolerable salt is acetate.

The GLP-1 agonist is preferably selected from the group consisting of exendin-4, analogs and derivates of exendin-4, and pharmacologically tolerable salts thereof.

A further preferred GLP-1 agonist is an analog of exendin-4 selected from the group consisting of:
H-desPro³⁶-exendin-4-Lys₆-NH₂,
H-des(Pro^{36, 37})-exendin-4-Lys₄-NH₂,
H-des(Pro^{36, 37})-exendin-4-Lys₅-NH₂ and pharmacologically tolerable salts thereof.

A further preferred GLP-1 agonist is an analog of exendin-4 selected from the group consisting of:
desPro³⁶[Asp²⁸]exendin-4(1-39),
desPro³⁶[IsoAsp²⁸]exendin-4 (1-39),
desPro³⁶[Met(O)¹⁴, Asp²⁸]exendin-4 (1-39),
desPro³⁶[Met(O)¹⁴, IsoAsp²⁸]exendin-4 (1-39),
desPro³⁶[Trp(O₂)²⁵, Asp²8]exendin-2 (1-39),
desPro³⁶[Trp(O₂)²⁵, IsoAsp²⁸]exendin-2 (1-39),
desPro³⁶[Met(O)¹⁴Trp(O₂)²⁵, Asp²⁸]exendin-4 (1-39),
desPro³⁶[Met(O)¹⁴Trp(O₂)²⁵, IsoAsp²⁸]exendin-4 (1-39) and pharmacologically tolerable salts thereof.

A further preferred GLP-1 agonist is an analog of exendin-4 selected from a group as described in the previous paragraph, wherein the peptide-Lys₆-NH₂ is attached to the C-termini of the analogs of exendin-4.

A further preferred GLP-1 agonist is an analog of exendin-4 selected from the group consisting of:
H-(Lys)₆-desPro³⁶[Asp²⁸]exendin-4(1-39)-Lys₆-NH₂,
desAsp²⁸Pro^{36,} Pro³⁷, Pro³⁸exendin-4 (1-39)-NH₂,
H-(Lys)₆-desPro³⁶, Pro³⁷, Pro³⁸[Asp²⁸]exendin-4(1-39)-NH₂,
H-Asn-(Glu)⁵desPro³⁶, Pro³⁷, Pro³⁸[Asp²⁸]exendin-4(1-39)-NH₂,
desPro^{36 36} Pro³⁷ Pro³⁸[Asp²⁰]exendin-4 (1-39)-(Lys)₆-NH₂,
H-(Lys)₆-desPro³⁶, Pro³⁷, Pro³⁸[Asp²⁸]exendin-4 (1-39)-(Lys)₆-NH₂,
H-Asn-(Glu)⁵-desPro³⁶, Pro³⁷, Pro³⁸[Asp²⁸]exendin-4 (1-39)-(Lys)₆-NH₂,
H-(Lys)₆-desPro³⁶[Trp(O₂)²⁵, Asp²⁸]exendin-4 (1-39)-Lys₆-NH₂,
H-desAsp²⁸ Pro³⁶, Pro³⁷, Pro³⁸[Trp(O₂)²⁵]exendin-4 (1-39)-NH₂,
H-(Lys)₆-desPro³⁶, Pro³⁷, Pro³⁸[Trp(O₂)²⁵, Asp²⁸]exendin-4(1-39)-NH₂,
H-Asn-(Glu)⁵-desPro³⁶, Pro³⁷, Pro³⁸[Trp(O₂)²⁵, Asp²⁸]exendin-4(1-39)-NH₂,
desPro³⁶Pro³⁷, Pro³⁸[Trp(O₂)²⁵ Asp²⁸]exendin-4 (1-39)-(Lys)₆-NH₂,
H-(Lys)₆-desPro³⁶, Pro³⁷, Pro³⁸[Trp(O₂)²⁵, Asp²⁸]exendin-4(1-39)-(Lys)₆-NH₂,
H-Asn-(Glu)₅-desPro³⁶, Pro³⁷, Pro³⁸[Trp(O₂)²⁵, Asp²⁸]exendin-4(1-39)-(Lys)₆-NH₂,
H-(Lys)₆-desPro³⁶[Met(O)¹⁴, Asp28]exendin-4 (1-39)-Lys₆-NH₂,
desMet(O)¹⁴ Asp28Pro 36, Pro37, Pro38exendin-4(1-39)-NH₂,
H-(Lys)₆-desPro³⁶, Pro ³⁷, Pro³⁸[Met(O)¹⁴, Asp²⁸]exendin-4(1-39)-NH₂,
H-Asn-(Glu)₅-desPro³⁶, Pro³⁷, Pro³⁸[Met(O)¹⁴, Asp²⁸]exendin-4(1-39)-NH₂,
desPro³⁶, Pro³⁷, Pro³⁸[Met(O)¹⁴, Asp²⁸]exendin-4 (1-39)-(Lys)₆-NH₂,
H-(Lys)₆-desPro³⁶, Pro³⁷, Pro³⁸[Met(O)¹⁴, Asp²⁸]exendin-4(1-39)-Lys₆-NH₂,
H-Asn-(Glu)₅-desPro³⁶, Pro³⁷, Pro³⁸[Met(O)¹⁴, Asp²⁸]exendin-4(1-39)-(Lys)₆-NH₂,
H-(Lys)₆-desPro³⁶[Met(O)¹⁴, Trp(O₂)^{25 25} Asp²⁸]exendin-4(1-39)-Lys₆-NH₂,
desAsp²⁸Pro³⁶, Pro³⁷, Pro³⁸[Met(O)¹⁴, Trp(O₂)²⁵]exendin-4(1-39)-NH₂,
H-(Lys)₆-desPro³⁶, Pro³⁷, Pro³⁸[Met(O)¹⁴, Trp(O₂)²⁵, Asp²⁸]exendin-4(1-39)-NH₂,
H-Asn-(Glu)₅-desPro³⁶, Pro³⁷, Pro³⁸[Met(O)¹⁴, Asp²⁸]exendin-4(1-39)-NH₂,
desPro³⁶, Pro³⁷, Pro³⁸[Met(O)¹⁴, Trp(O₂)²⁵, Asp²⁸]exendin-4(1-39)-(Lys)₆-NH₂,
H-(Lys)₆-desPro³⁶, Pro³⁷, Pro³⁸[Met(O)¹⁴, Trp(O₂)²⁵, Asp²⁸]exendin-4(1-39)-(Lys)₆-NH₂,
H-Asn-(Glu)₅-desPro³⁶, Pro³⁷, Pro³⁸[Met(O)¹⁴, Trp(O₂)²⁵, Asp²⁸]exendin-4(1-39)-(Lys)₆-NH₂ and pharmacologically tolerable salts thereof.

Likewise, the GLP-1 agonist can be selected from the group consisting of GLP-1 and analogs and derivates of GLP-1. A further preferred GLP-1 agonist is selected from the group consisting of Arg³⁴, Lys²⁶ (N^{ε}(γ-glutamyl (N^{α}-hexadecanoyl)))GLP-1 (7-37) [liraglutide] and a pharmacologically tolerable salt thereof.

A further preferred GLP-1 agonist is AVE0010. AVE0010 has the sequence desPro³⁶exendin-4 (1-39)-Lys₆-NH₂ (SEQ ID NO:1). Likewise, pharmacologically tolerable salts of AVE0010 are preferred.

The GLP-1 agonist, for example AVE0010, is more particularly used in an amount ranging from 0.01 mg/ml to 0.5 mg/ml or 0.05 mg/ml to 1.5 mg/ml.

In the present application, the term “insulin” encompasses not only unmodified insulins but also insulin analogs, insulin derivatives, and insulin metabolites. The compositions according to the present invention comprise one or more independently selected from the group consisting of insulins (e.g., unmodified insulins), insulin analogs, insulin derivatives, and insulin metabolites, and any desired combinations thereof.

The at least one insulin may independently be selected from the group consisting of bovine insulins, analogs, derivatives, and metabolites thereof, porcine insulins, analogs, derivatives, and metabolites thereof, and human insulins, analogs, derivatives, and metabolites thereof. Preferably, the at least one insulin is independently selected from human insulins, analogs, derivatives, and metabolites thereof.

Furthermore, an insulin according to the present invention may be selected independently from unmodified insulins, more particularly from bovine insulins, porcine insulins, and human insulins.

The at least one insulin may independently be selected from the group consisting of bovine insulins, porcine insulins, and human insulins. More preferably, the at least one insulin is independently selected from human insulins. An insulin according to the present invention may be selected from unmodified insulins, more particularly from bovine insulins, porcine insulins, and human insulins.

Insulin derivatives according to the present invention are derivatives of a naturally occurring insulin and/or an insulin analog, which are obtained by chemical modification. The chemical modification may consist, for example, in the addition of one or more defined chemical groups onto one or more amino acids.

Insulin analogs which are described in EP 0 214 826, EP 0 375 437, EP 0 678 522, EP 0 419 504, WO 92/00321, EP-A 0 368 187, and WO2009/063072 may be part of the compositions according to the present invention. The documents EP 0 214 826, EP 0 375 437, EP 0 678 522, EP 0 419 504, WO 92/00321, EP-A 0 368 187, and WO 2009/063072 are included herein by reference.

One preferred insulin analog according to the present invention may be selected from the group consisting of Gly (A21)-Arg(B31)-Arg(B32) human insulin (insulin glargine), Lys(B3)-Glu(B29) human insulin; Lys^{B28}Pro^{B29} human insulin (insulin lyspro), B28 Asp human insulin (insulin aspart), human insulin in which proline in position B28 has been substituted by Asp, Lys, Leu, Val or Ala and where Lys in position B29 may be substituted by Pro; AlaB26 human insulin; des(B28-B30) human insulin; des(B27) human insulin or B29Lys(ε-tetradecanoyl), des(B30) human insulin (insulin detemir), N^{εB29}-tetradecanoyl des(B30) human insulin, N^{εB29}-(N^{α}-(HOOC(CH₂)₁₄CO)-γ-Glu) desB30 human insulin, Lys^{B29} (N^{ε} lithocholyl-γ-Glu)-des(B30) human insulin, N^{εB29}-ω-carboxypentadecanoyl-γ-L-glutaylamide desB30 human insulin, and N^{εB29}-ω-carboxypentadecanoyl-γ-amino-butanoyl des(B30) human insulin.

A preferred insulin derivative according to the present invention may be selected from the group consisting of B29-N-myristoyl-des (B30) human insulin, B29-N-palmitoyl-des(B30) human insulin, B29-N-myristoyl human insulin, B29-N-palmitoyl human insulin, B28-N-myristoyl Lys^{B28}Pro^{B29} human insulin, B28-N-palmitoyl-Lys^{B28}Pro^{B29} human insulin, B30-N-myristoyl-Thr^{B29}Lys^{B30} human insulin, B30-N-palmitoyl-Thr^{B29}Lys^{B30} human insulin, B29-N-(N-palmitoyl-Y-glutamyl)-des(B30) human insulin, B29-N-(N-lithocholyl-Y-glutamyl)-des(B30) human insulin, B29-N-(ω-carboxyheptadecanoyl)-des(B30) human insulin and B29-N-(ω-carboxyheptadecanoyl) human insulin, N^{εB29}-tetradecanoyl des(B30) human insulin, N^{εB29}-(N^{α}-(HOOC(CH₂)₁₄CO)-γ-Glu) des B30 human insulin, Lys^{B29} (N^{ε} lithocholyl-γ-Glu)-des (B30) human insulin, N^{εB29}-ω-carboxypentadecanoyl-γ-L-glutaylamide desB30 human insulin, and N^{εB29}-ω-carboxypentadecanoyl-γ-amino-butanoyl des(B30) human insulin.

A more highly preferred insulin derivative according to the present invention is selected from the group consisting of Gly(A21)-Arg(B31)-Arg(B32) human insulin, Lys^{B28}Pro^{B29} human insulin (insulin lyspro), B28 Asp human insulin (insulin aspart), B29Lys(ε-tetradecanoyl), desB30 human insulin (insulin detemir).

The compositions according to the present invention contain 60-6000 nmol/ml, preferably 240-3000 nmol/ml, of an insulin as defined herein. Depending on the insulin used, a concentration of 240-3000 nmol/ml corresponds approximately to a concentration of 1.4-35 mg/ml or 40-500 units/ml.

The present invention particularly preferably provides a composition as described herein comprising at least one insulin independently selected from Lys^{B28}Pro^{B29} human insulin (insulin lyspro), B28 Asp human insulin (insulin aspart), B29Lys(ε-tetradecanoyl), desB30 human insulin (insulin detemir), and insulin glargine (Gly(A21)-Arg(B31)-Arg(B32) human insulin), and comprising AVE0010 and/or a pharmacologically tolerable salt thereof. The present invention further particularly preferably provides a composition as described herein comprising insulin glargine (Gly(A21)-Arg(B31)-Arg(B32) human insulin) and AVE0010 (desPro³⁶exendin-4 (1-39)-Lys₆-NH₂) and/or a pharmacologically tolerable salt thereof. These particularly preferred compositions preferably have an acidic pH of 1-6.8, more preferably pH 3.5-6.8, even more preferably pH 3.5-4.5.

In a particular embodiment, the formulation according to the present invention comprises the following constituents:
(a) desPro³⁶exendin-4 (1-39)-Lys₆-NH₂,
(b) Gly(A21)-Arg(B31)-Arg(B32) human insulin,
(c) zinc chloride,
(d) m-cresol,
(e) L-methionine,
(f) glycerol,
(g) hydrochloric acid, if adjustment to a pH of approximately 4.5 is required,
(h) NaOH solution, if adjustment to a pH of approximately 4.5 is required, and
(i) water.

More particularly, the formulation according to the present invention consists of the constituents mentioned in (a) to (i). Optionally, m-cresol can be omitted. Hence the formulation according to the present invention then consists of constituents (a) to (c) and (e) to (i).

The present invention further provides a combination of at least two formulations according to the present invention. In this case, a first and a second composition and, optionally, at least one further pharmaceutical composition are provided, each comprising the insulin and the GLP-1 agonist.

Therefore, the present invention provides a combination comprising a first pharmaceutical composition and a second pharmaceutical composition, and, optionally, at least one further pharmaceutical composition, each comprising at least one insulin and at least one GLP-1 agonist, and containing the at least one insulin and/or the at least one GLP-1 agonist in different weight fractions relative to the total weight of the composition.

In the present application, “optionally, at least one further pharmaceutical composition” means that the combination according to the present invention, in addition to the first and second pharmaceutical compositions, may comprise at least one further pharmaceutical composition. Hence, the combination according to the present invention may comprise, for example, 3, 4, 5, 6, 7, 8, 9, 10 or more pharmaceutical compositions according to the present invention.

Preferred combinations are those which comprise a first and a second pharmaceutical composition according to the present invention.

Likewise preferred are combinations which comprise a first, a second, and a third pharmaceutical composition according to the present invention.

Likewise preferred are combinations which comprise a first, a second, a third, and a fourth pharmaceutical composition according to the present invention.

Likewise preferred are combinations which comprise a first, a second, a third, a fourth, and a fifth pharmaceutical composition.

The weight fractions of the at least one insulin and of the at least one GLP-1 agonist may be selected in the first pharmaceutical composition, the second pharmaceutical composition, and, where used, the at least one further pharmaceutical composition in such a way that the pharmaceutical compositions contain different ratios of insulin to GLP-1 agonist, based on the weight fraction.

In this case, the first composition may contain the smallest ratio and the second composition the next-greater ratio. Where at least one further composition is present, it may contain the next-greater ratio. Where a further composition is present as well, it may contain the next-greater ratio in turn. The compositions may therefore contain ratios of insulin to GLP-1 agonist, based on the weight fraction, that increase from the first to the second and, where used, further compositions.

The weight fraction of one of the two active ingredients, i.e., of the at least one insulin or of the at least one GLP-1 agonist, in the first pharmaceutical composition, the second pharmaceutical composition, and, where used, the at least one further pharmaceutical composition is preferably selected in each case such that the predetermined dose of this active ingredient can be administered by administering a defined volume of the first, second and/or at least one further composition. With particular preference, this active ingredient is the at least one insulin.

The weight fraction of the other of the two active ingredients, i.e., of the at least one insulin or of the at least one GLP-1 agonist, in the first pharmaceutical composition, the second pharmaceutical composition, and, where used, the at least one further pharmaceutical composition is preferably selected such that the ratios of insulin to GLP-1 agonist, based on the weight fraction, increase from the first to the second and, where used, further compositions. With particular preference, this active ingredient is the at least one GLP-1 agonist.

Furthermore, the weight fraction of the other of the two active ingredients in the pharmaceutical compositions is determined such that one of the pharmaceutical compositions can be selected in such a way that the dose of the first of the two active ingredients that is to be administered and the dose of the second active ingredient that is to be administered are given in a defined volume. Hence, a pharmaceutical composition is selected which contains the desired ratio.

Theoretically, it would be possible to provide a pharmaceutical composition for each individual therapeutically desired ratio of the weight fractions of the at least one insulin to the at least one GLP-1 agonist, in order to obtain an optimum dosage, tailored to requirements, for both active ingredients for every patient.

In the present invention, a particular number of pharmaceutical compositions is sufficient in order to cover the dosages needed in practice for the two active ingredients. For each patient, a defined dosage range is defined within a therapeutically rational interval for each of the two active ingredients. The dose to be administered ought hereby to fluctuate essentially within this dosage range for a particular patient, without any overdosing or underdosing.

Since it is primarily the amount of insulin that must be adapted and precisely dosed to the individual patient, the concentration range of the GLP-1 agonist allows a pharmaceutical composition according to the present invention that contains a defined ratio of at least one insulin to the at least one GLP-1 agonist to cover a therapeutic range of insulin doses simultaneously with the associated, synergistic amount of GLP-1 agonist. The ratio can be selected such that every desired insulin dose has its corresponding dose of the at least one GLP-1 agonist, which is situated within the desired range, e.g., the synergistic range. As set out earlier on above, the ratios of the first, second, and, where used, at least one further composition of the pharmaceutical may also be chosen such that the ratios increase from the first to the second and, where used, the at least one further composition. If the dose of the GLP-1 agonist at the desired insulin dose of a composition (e.g., of the first composition) is outside (generally above) the desired dosage range of the GLP-1 agonist, then the next composition (e.g., the second composition) or a further composition with a greater ratio of the at least one insulin to the at least one GLP-1 agonist is selected for use, in which the amount of the GLP-1 agonist at the desired insulin dose lies within the desired range. The ratios of the first, second, and, where used, at least one further composition of the combination may further be chosen such that the ranges of the insulin dosages which correspond to the desired dosages of the at least one GLP-1 agonist border one another and/or overlap one another. Preferably, the ranges overlap. Overlapping means more particularly that it is possible to select at least two compositions which, at the desired dose of the at least one insulin, each contain an amount of the at least one GLP-1 agonist which lies within the desired dosage range.

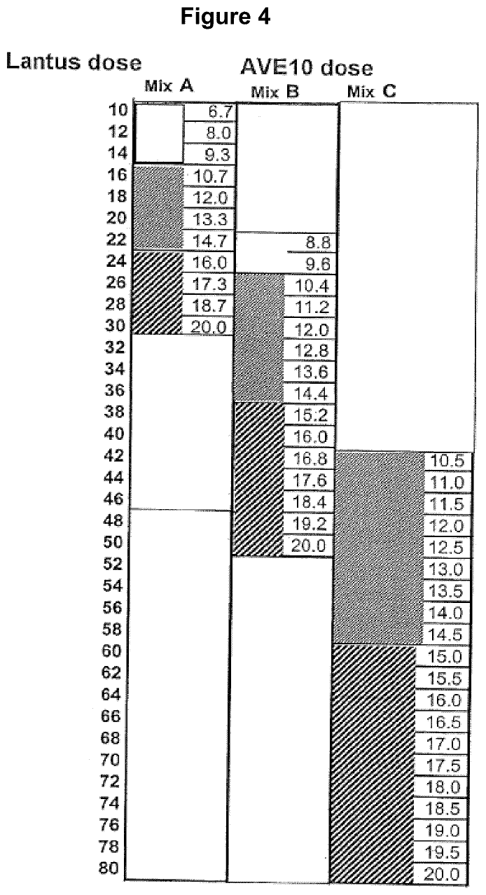

For example, 3 compositions are sufficient to adjust the dose of the at least one insulin for an individual patient to a level selected from the range from 15 to 80 units of insulin and at the same time to dose the GLP-1 agonist with an amount within the range from 10 to 20 μg (see FIG. 4).

It is also possible to provide a combination according to the present invention in which the ratio is selected such that for each desired dosage of the GLP-1 agonist there is a corresponding dosage of the at least one insulin which lies within the desired range. The ratios of the first, second, and, where used, at least one further composition of the pharmaceutical may also be chosen such that the ranges of the dosages of the GLP-1 agonist that correspond to the desired dosages of the at least one insulin border one another and/or overlap one another. Preferably, the ranges overlap. Overlapping in this context means more particularly that it is possible to select at least two compositions which, at the desired dosage of the at least one GLP-1 agonist, each contain an amount of the at least one insulin that lies within the desired dosage range.

Preferably, the combination according to the present invention contains not more than 10 pharmaceutical compositions as defined above, more preferably not more than 5, not more than 4, not more than 3 or 2 pharmaceutical compositions.

The compositions according to the present invention may contain the at least one GLP-1 agonist in, in each case, identical or different weight fractions. For example, at least two of the compositions according to the present invention may contain the at least one GLP-1 agonist in a substantially identical weight fraction.

It is preferred for the first, second, and, where used, further composition(s) to contain the at least one GLP-1 agonist in a substantially identical weight fraction and the at least one insulin in different weight fractions.

The compositions according to the present invention may, however, also contain the at least one insulin in, in each case, identical or different weight fractions. For example, at least two of the compositions according to the present invention may contain the at least one insulin in a substantially identical weight fraction.

It is especially preferred for the first, second, and, where used, further composition(s) to contain the at least one insulin in a substantially identical weight fraction and the at least one GLP-1 agonist in different weight fractions.

A first preferred composition according to the present invention comprises:

| | | |
|---|---|---|
| (a) AVE0010 | approximately 0.025 | mg |
| (b) insulin glargine | approximately 3.64 | mg |
| (c) zinc chloride | approximately 0.06 | mg |
| (d) 85% glycerol | approximately 20.0 | mg |
| (e) m-cresol | approximately 2.7 | mg |
| (f) L-methionine | approximately 3.0 | mg |
| (g) NaOH | q.s. pH 4.5 | |
| (h) HCl, 36% | q.s. pH 4.5 | |
| (i) water | ad 1 | mL |

A second preferred composition according to the present invention comprises:

| | | |
|---|---|---|
| (a) AVE0010 | approximately 0.04 | mg |
| (b) insulin glargine | approximately 3.64 | mg |
| (c) zinc chloride | approximately 0.06 | mg |
| (d) 85% glycerol | approximately 20.0 | mg |
| (e) m-cresol | approximately 2.7 | mg |
| (f) L-methionine | approximately 3.0 | mg |
| (g) NaOH | q.s. pH 4.5 | |
| (h) HCl, 36% | q.s. pH 4.5 | |
| (i) water | ad 1 | mL |

A third preferred composition according to the present invention comprises:

| | | |
|---|---|---|
| (a) AVE0010 | approximately 0.066 | mg |
| (b) insulin glargine | approximately 3.64 | mg |
| (c) zinc chloride | approximately 0.06 | mg |
| (d) 85% glycerol | approximately 20.0 | mg |
| (e) m-cresol | approximately 2.7 | mg |
| (f) L-methionine | approximately 3.0 | mg |
| (g) NaOH | q.s. pH 4.5 | |
| (h) HCl, 36% | q.s. pH 4.5 | |
| (i) water | ad 1 | mL |

A fourth preferred composition according to the present invention comprises:

| | | |
|---|---|---|
| (a) AVE0010 | approximately 0.1 | mg |
| (b) insulin glargine | approximately 3.64 | mg |
| (c) zinc chloride | approximately 0.06 | mg |
| (d) 85% glycerol | approximately 20.0 | mg |
| (e) m-cresol | approximately 2.7 | mg |
| (f) L-methionine | approximately 3.0 | mg |
| (g) NaOH | q.s. pH 4.5 | |
| (h) HCl, 36% | q.s. pH 4.5 | |
| (i) water | ad 1 | mL |

Especially preferred is a combination comprising at least 2, 3, or 4 of the first, second, third, and fourth preferred composition mentioned.

In the present application, “approximately” means that the constituents can be present, for example, within the ranges of ±10, ±20, or ±30 around the specified values in the compositions according to the present invention or/and the combinations; preference is give to ±10.

When the composition according to the present invention or the combination comprises more than one insulin, these insulins are selected independently of one another.

When the composition according to the present invention or the combination comprises more than one GLP-1 agonist, these GLP-1 agonists are selected independently of one another.

The combination according to the present invention is provided more particularly as a pharmaceutical.

The present invention additionally provides a kit comprising a combination according to the present invention comprising at least one, not more than four, composition(s) according to the present invention and also, optionally, Lantus®. The kit according to the present invention may be intended for use by medical staff or by persons without specialist medical training, more particularly by the patients themselves or helpers such as relatives. In the kit according to the present invention, the individual pharmaceutical compositions comprising the combination according to the present invention are assembled in separate packs, and so the patient is able to select the composition appropriate to the current requirement and to administer an amount in line with that requirement. The kit according to the present invention comprises, for example, the combination according to the present invention in the form of a set of syringes, glass ampoules, and/or pens which contain at least one of the compositions according to the present invention, optionally in combination with the composition of Lantus®.

Suitable packaging is a syringe or a glass vessel with a suitable closure, from which individual therapeutically effective doses can be withdrawn as needed. Equally suitable are injection pens for administering insulin; such pens comprise a container (e.g. a cartridge) which contains a pharmaceutical composition according to the present invention.

More particularly, the kit according to the present invention is an injection pen consisting of two separate containers from which, in each case, individual therapeutic doses can be withdrawn as needed. Equally, the kit is a syringe consisting of two containers in which the second container is equipped as a reservoir needle.

In the present invention, the kit preferably consists of a combination of a first formulation, which comprises the GLP-1 agonist, an insulin, glycerol, zinc chloride, optionally m-cresol, L-methionine at a pH of 4.5 in water, and a second formulation, which preferably comprises an insulin, glycerol, zinc chloride, and m-cresol at a pH of 4.5 in water.

The first formulation may preferably have the following composition:

| | | | |
|---|---|---|---|
| (a) AVE0010 | approximately 0.4 | mg | or approximately 0.8 mg |
| (b) insulin glargine | approximately 3.64 | mg | |
| (c) zinc chloride | approximately 0.06 | mg | |
| (d) 85% glycerol | approximately 20.0 | mg | |
| (e) m-cresol | 0.0 | mg | or approximately 2.7 mg |
| (f) L-methionine | approximately 3.0 | mg | |
| (g) NaOH | q.s. pH 4.5 | | |
| (h) HCl, 36% | q.s. pH 4.5 | | |
| (i) water | ad 1 | ml. | |

The second formulation may preferably have the following composition:

| | | |
|---|---|---|
| (a) insulin glargine | approximately 3.64 | mg |
| (b) zinc chloride | approximately 0.06 | mg |
| (c) 85% glycerol | approximately 20.0 | mg |
| (d) m-cresol | approximately 2.7 | mg |
| (e) NaOH | q.s. pH 4.5 | |
| (f) HCl, 36% | q.s. pH 4.5 | |
| (g) water | ad 1 | ml. |

The present invention further provides for a method for treating a patient with a composition according to the present invention, comprising administering the composition to the patient.

The present invention yet further provides a method for treating a patient with a combination according to the present invention or with a kit as described herein. More particularly, this method comprises the administration of a combination according to the present invention comprising a first pharmaceutical composition and a second pharmaceutical composition, and, optionally, at least one further pharmaceutical composition, each comprising at least one insulin and at least one GLP-1 agonist, and comprising the at least one insulin and/or the at least one GLP-1 agonist in different weight fractions relative to the total weight of the composition, said method comprising:
(a) selecting a dose of the at least one insulin that is to be administered,
(b) selecting a dose of the at least one GLP-1 agonist that is to be administered,
(c) selecting a composition from the first, second, and, where used, at least one further composition of the pharmaceutical that comprises the doses from (a) and (b) in a concentration such that the doses from (a) and (b) are present in the same volume, and
(d) determining and administering an amount which corresponds to the doses from (a) and (b).

The dose according to step (a) and/or step (b) is determined according to the individual requirement of the patients.

Step (c) of the treatment method according to the present invention can be carried out by referring to a table. This table may be part of the combination according to the present invention, of the pharmaceutical according to the present invention, or of the kit according to the present invention. Example 2 contains an example of a table according to the present invention.

The composition according to the present invention, the combination according to the present invention, the pharmaceutical according to the present invention, or/and the kit according to the present invention is/are intended more particularly for treating diabetes mellitus, more particularly for treating type I or type II diabetes mellitus. Further possible indications are symptoms which are associated with diabetes mellitus. Preferably, the composition according to the present invention is used to control the fasting, postprandial, or/and postabsorptive plasma glucose concentration, to improve glucose tolerance, to prevent hypoglycemia, to prevent functional loss of the B-cells of the pancreas, to effect weight loss, or/and to prevent weight gain.

The present invention further provides for the use of a composition according to the present invention, a combination according to the present invention, or a kit according to the present invention in the manufacture of a pharmaceutical for treating diabetes mellitus, more particularly type I or type II, or/and the symptoms associated with it, as described herein.

The present invention further provides a method for manufacturing a composition according to the present invention, a combination according to the present invention, or/and a kit according to the present invention, comprising formulating a GLP-1 agonist or/and a pharmacologically tolerable salt thereof with an insulin or/and a pharmaceutically acceptable salt thereof, methionine, and, optionally, at least one pharmaceutically acceptable excipient.

The present invention further provides a method for manufacturing a composition according to the present invention, comprising formulating a GLP-1 agonist or/and a pharmacologically tolerable salt thereof with methionine and, optionally, at least one pharmaceutically acceptable excipient.

The present invention further provides for the use of the compositions according to the invention together with the administration of metformin, insulin glargine, or AVE0010, more particularly in an add-on therapy for administering metformin, insulin glargine, or AVE0010.

More particularly, the composition comprises des Pro³⁶ exendin-4(1-39)-Lys₆-NH₂ (AVE0010) and/or a pharmacologically tolerable salt thereof, insulin glargine and/or a pharmacologically tolerable salt thereof.

Especially preferred is the add-on therapy of the preferred composition in type II diabetes patients who cannot be sufficiently controlled with insulin glargine and/or AVE0010. Also contemplated are patients who are younger than 50 years and/or have a body mass index of at least 30.

In the present invention, the add-on therapy involves more particularly the treatment of type II diabetes with the composition according to the present invention as a supplement to metformin, AVE0010, and/or insulin glargine. The composition according to the present invention can be added in a time interval of 24 hours (once-a-day dosage). Metformin, insulin glargine, and AVE0010 can be administered by means of different routes of administration. Metformin can be administered orally, AVE0010 and insulin glargine, in each case, subcutaneously.

Patients treated with the add-on therapy according to the present invention can have an HbA1c value in the range of 7% to 10%. They are preferably in the age range of 18 to 50 years.

The use in the add-on therapy according to the present invention is more particularly applicable to patients in whom type II diabetes cannot be sufficiently controlled with metformin, AVE0010, or insulin glargine alone. The therapy is preferred in the case of insufficient control through insulin glargine or AVE0010.

The present invention further provides for the use of the composition according to the present invention as a supplement to a diet in order to control the blood sugar level in type II diabetes patients when the application of insulin glargine and AVE0010 is indicated.

More particularly, metformin is administered as follows: at least 1.0 g/day, preferably at least 1.5 g/day for 3 months.

The invention is further elucidated by the following figures and examples.

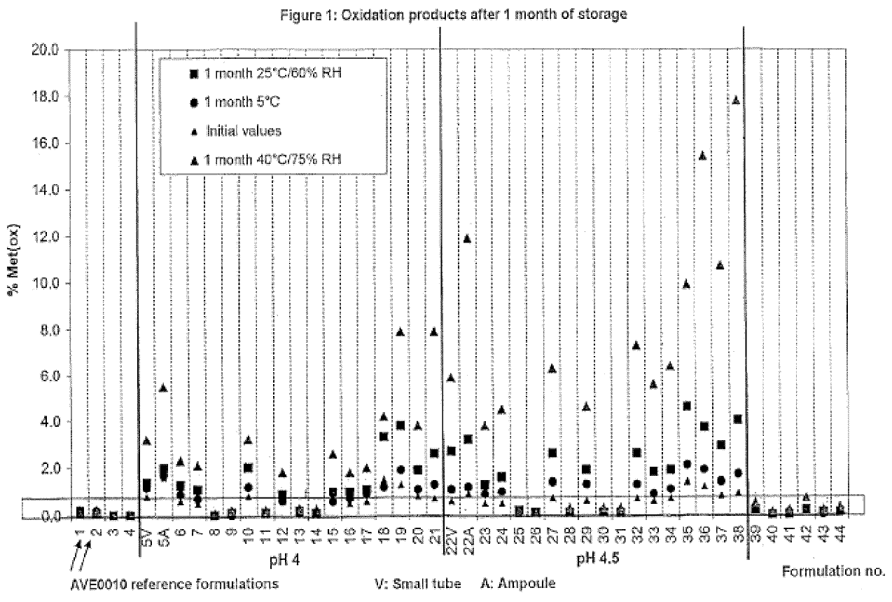

FIG. 1 shows the content of oxidized methionine Met(ox) in AVE0010 after 1 month of storage at different temperatures relative to the start of storage. The frame shows the values for the AVE0010 reference formulation no. 1 and 2.

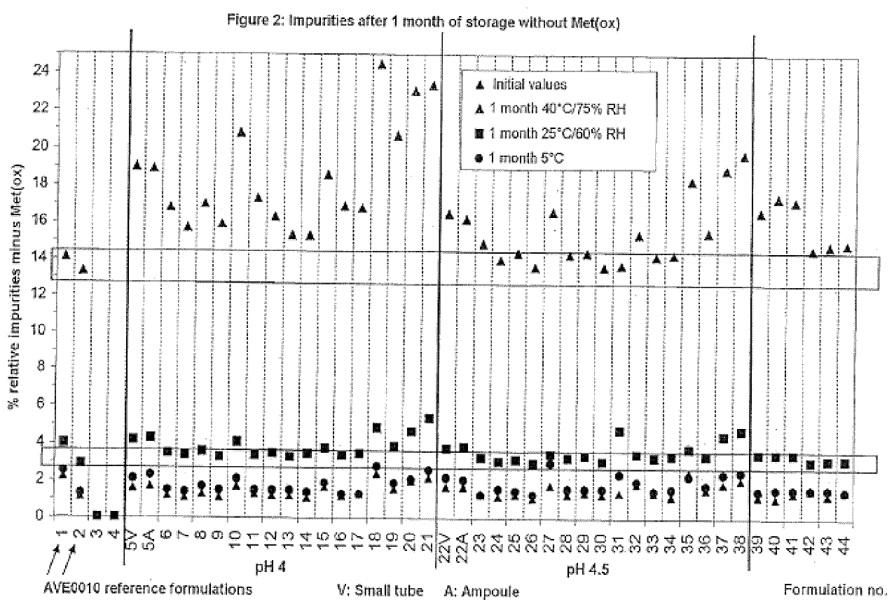

FIG. 2 shows the content of impurities of AVE0010 without Met(ox) after 1 month of storage at different temperatures relative to the start of storage.

The frames show the values of the AVE0010 reference formulations at 25° C. and at 40° C.

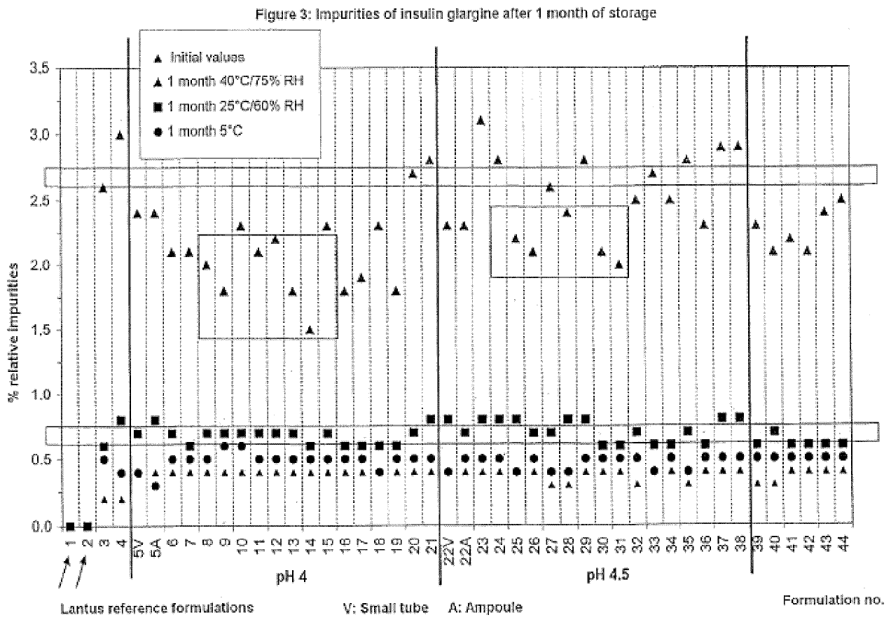

FIG. 3 shows the content of impurities of insulin glargine after 1 month of storage at different temperatures relative to the start of storage. The narrow frames show the values of the insulin glargine reference formulations at 25° C. and at 40° C. The broad frames indicate the formulations having the lowest fractions of AVE0010 impurities.

FIG. 4: the “3 pens cover all” concept.

### EXAMPLE 1

### 1. Purpose of Study

The physical and chemical stability of compositions comprising a GLP-1 agonist (AVE0010) and an insulin (insulin glargine, Lantus) was tested.

### 2. Formulations Used

For the formulations tested, the substances were used in the following concentrations/amounts:

| | | | | Amount |
|---|---|---|---|---|
| | | | | used |
| Substance | Pharmacopeia | Manufacturer | Designation | [mg/mL] |
| Insulin | | Sanofi-Aventis | | 3.63 |
| glargine | | | | 7.27 |
| | | | | 10.67 |
| AVE0010 | | Poly Peptide | | 0.1 |
| | | LabTorrance | | 0.025 |
| | | CA, USA | | |
| Methionine | USP | MP | | 3 |
| | | Biomedicals | | |
| Zinc chloride | Ph. Eur., | Merck | | 0.03 |
| | USP, BP | | | 0.06 |
| | | | | 0.09 |
| Glycerol, 85% | Ph. Eur., JP | Hedinger, | | 20 |
| | | Stuttgart | | 18 |
| m-Cresol | Ph. Eur., USP | Hedinger, | | 2.7 |
| | | Stuttgart | | |
| Polysorbate 20 | Ph. Eur., JP | Kolb | Tween 20 | 0.02 |
| Polysorbate 80 | Ph. Eur. | SEPPIC | Tween 80 | 0.02 |
| Poloxamer 188 | | BASF, | Lutrol F68 | 0.02 |
| | | Ludwigshafen | | |
| Benzalkonium | Ph. Eur., JP | Sigma-Aldrich | | 0.02 |
| chloride | | | | |
| L-Lysine | | Resum, | | 1.0 |
| | | F-Ham, | | 5.0 |
| | | Degussa | | |
| Acetate | | | | 1.75 |
| | | | | 3.5 |
| NaOH | Ph. Eur., JP | Merck | | 0.1N, for |
| | | | | adjusting |
| | | | | to |
| | | | | pH 4.0 or |
| | | | | 4.5 |
| HCl | Ph. Eur., JP | Merck | | 0.1N, for |
| | | | | adjusting |
| | | | | to |
| | | | | pH 4.0 or |
| | | | | 4.5 |
| Wfl | | | | Ad 1 mL |

When a factor is mentioned in conjunction with a constituent of a formulation (e.g., ½, ¼, 2×, 3×, 5×, as in ½ acetate, 5× lysine, 2× Lantus, and 3× Lantus), the concentrations of the substance concerned were used at a reduced or increased concentration depending on the factor.

### 3. Test Method

### 3.1 Physical Stability

### 3.1.1 THT Test

Thioflavin T (THT) binds specifically to protein fibrils, which leads to a change in THT fluorescence. THT does not bind to AVE0010 or insulin. The kinetics of fibril formation can be measured in the presence of THT as the change in fluorescence. An increase in fluorescence corresponds to fibril formation. The shape of the curves allows conclusions about the tendency of a formulation to form fibrils.

Fluorescence measurements were carried out on a Tecan Infinite 200 fluorescence measurement instrument. For analysis of fibrillation kinetics, a Photomed FluoDia 770 high-temperature fluorescence microplate reader was used. The thioflavin T fluorescence spectra were carried out with a Tecan Infinite 200 fluorescence measurement instrument at 23° C. Insulin (900 μl) was mixed with 10 μl of thioflavin T (1 mM in H₂O). The mixture was then distributed into a black V-shaped 96-well plate from Biozym (100 μl per well). The emission of fluorescence was measured between 470 and 600 nm (in increments of 1 nm) after excitation at 450 nm with a gain of 100, an integration time of 200 μs, and 25 readings at room temperature.

The binding kinetics of thioflavin T were measured on a Photomed FluoDia 770 high-temperature fluorescence microplate reader. The instrument consists essentially of a 50 W quartz halogen lamp for excitation, filter wheels for excitation and emission which can each contain up to 4 filter sets, and a PMT detector. The heating plate for 96-well plates allows very high precision with regard to temperature (better than ±0.3° C.).

A solution (10 μl) of thioflavin T (10.1 mM in ultrapure water) was added to 1 ml of the formulations and gently mixed by inverting the small tubes several times. The mixture was then distributed into a black V-shaped 96-well plate from Biozym (100 μl per well, 8 wells per sample). All measurements were carried out with the following parameters:
Number of cycles: 181
Excitation filter: 450 nm
Interval: 1 min
Emission filter: 486 nm
Integration time: 20 ms
Temperature control: Standard temperature-control mode
Number of averagings: 4
Target temperature: 70° C.
Attenuation: 4

Fluorescence mean values were determined from 8 parallel measurements.

### 3.2 Chemical Stability

The formulations were tested for chemical stability after preparation (t0) or after storage for 1 month at 4° C., 25° C. (60% relative humidity), and 40° C. (75% relative humidity). The measurements were carried out on an HPLC instrument (model: alliance) from Water Systems, using the 100% peak area method. For separation, a gradient of 0.1% TFA and acetonitrile as the mobile phase and a C18 reversed-phase column (Jupiter) as the stationary phase were used. For analysis, the formulation was treated with a zinc acetate solution, which led to precipitation of insulin glargine. The precipitates were centrifuged down, and only the supernatant was analyzed.

Impurities of insulin glargine: the amount of impurities was determined with an HPLC (Water Systems), using the 100% peak area method. For separation, a sodium phosphate-buffered solution (pH 2.5) with NaCl and acetonitrile gradients was used as the mobile phase. A C18 reversed-phase column (Supersher) was used as the stationary phase.

### 4. Summary of Experimental Data on Physical Stability

| | | | | THT |
|---|---|---|---|---|
| | | | | 3 h, 70° C. |
| | | | | relative |
| | | | | fluorescence |
| Formulation | | | | intensity |
| No. | Batch | Composition | pH | at 486 nm |
| 1 | 630 | AVE0010 standard, | 4.5 | 536 |
| | | industrial scale | | |
| 2 | 567 | AVE0010 standard, | 4 | 518 |
| | | fresh | | |
| 3 | 631 | Lantus standard, | 4.0 | 2952 |
| | | industrial scale | | |
| 4 | 560 | Lantus standard, fresh | 4 | 1566 |
| 5 | 568 | Lantus form., AVE0010 | 4 | 2037 |
| 6 | 569 | Lantus form., AVE0010, | 4 | 11763 |
| | | ½ acetate buffer | | |
| 7 | 570 | Lantus form., AVE0010, | 4 | 69184 |
| | | acetate buffer | | |
| 8 | 582 | Lantus form., AVE0010, | 4 | 2053 |
| | | methionine | | |
| 9 | 583 | Lantus form., AVE0010, | 4 | 18814 |
| | | 1/2 acetate buffer, | | |
| | | methionine | | |
| 10 | 584 | Lantus form., AVE0010, | 4 | 8183 |
| | | polysorbate 20 | | |
| 11 | 585 | Lantus form., AVE0010, | 4 | 6731 |
| | | polysorbate 20, | | |
| | | methionine | | |
| 12 | 586 | Lantus form., AVE0010, | 4 | 13897 |
| | | polysorbate 20, | | |
| | | ½ acetate buffer | | |
| 13 | 587 | Lantus form., AVE0010, | 4 | 22200 |
| | | polysorbate 20, | | |
| | | ½ acetate buffer, | | |
| | | methionine | | |
| 14 | 588 | Lantus form., AVE0010, | 4 | 134093 |
| | | polysorbate 20, | | |
| | | acetate buffer, | | |
| | | methionine | | |
| 15 | 590 | Lantus form., AVE0010, | 4 | 3362 |
| | | lysine | | |
| 16 | 591 | Lantus form., AVE0010, | 4 | 19677 |
| | | lysine, | | |
| | | ½ acetate buffer | | |
| 17 | 592 | Lantus form., AVE0010, | 4 | 30176 |
| | | lysine, | | |
| | | ½ acetate buffer, | | |
| | | polysorbate 20 | | |
| 18 | 593 | Lantus form., | 4 | 3107 |
| | | ¼ AVE0010 | | |
| 19 | 594 | Lantus form., | 4 | 74662 |
| | | ¼ AVE0010, | | |
| | | 5 × lysine | | |
| 20 | 595 | 2 × Lantus AVE0010 | 4 | 4504 |
| 21 | 596 | 3 × Lantus AVE0010 | 4 | 30251 |
| 22 | 604 | Lantus form., AVE0010 | 4.5 | 4357 |
| 23 | 605 | Lantus form., AVE0010, | 4.5 | 36338 |
| | | ½ acetate buffer | | |
| 24 | 606 | Lantus form., AVE0010, | 4.5 | 72370 |
| | | acetate buffer | | |
| 25 | 607 | Lantus form., AVE0010, | 4.5 | 5429 |
| | | methionine | | |
| 26 | 608 | Lantus form., AVE0010, | 4.5 | 34714 |
| | | ½ acetate buffer, | | |
| | | methionine | | |
| 27 | 609 | Lantus form., AVE0010, | 4.5 | 1166 |
| | | polysorbate 20 | | |
| 28 | 610 | Lantus form., AVE0010, | 4.5 | 5564 |
| | | polysorbate 20, | | |
| | | methionine | | |
| 29 | 611 | Lantus form., AVE0010, | 4.5 | 12115 |
| | | polysorbate 20, | | |
| | | ½ acetate buffer | | |
| 30 | 612 | Lantus form., AVE0010, | 4.5 | 16397 |
| | | polysorbate 20, | | |
| | | ½ acetate buffer, | | |
| | | methionine | | |
| 31 | 613 | Lantus form., AVE0010, | 4.5 | 779 |
| | | polysorbate 20, | | |
| | | acetate buffer, | | |
| | | methionine | | |
| 32 | 614 | Lantus form., AVE0010, | 4.5 | 9726 |
| | | lysine | | |
| 33 | 615 | Lantus form., AVE0010, | 4.5 | 74027 |
| | | lysine, | | |
| | | ½ acetate buffer | | |
| 34 | 616 | Lantus form., AVE0010, | 4.5 | 9520 |
| | | lysine, | | |
| | | ½ acetate buffer, | | |
| | | polysorbate 20 | | |
| 35 | 617 | Lantus form., | 4.5 | 3713 |
| | | ¼ × AVE0010 | | |
| 36 | 618 | Lantus form., | 4.5 | 83384 |
| | | ¼ × AVE0010, | | |
| | | 5 × lysine | | |
| 37 | 619 | 2 × Lantus AVE0010 | 4.5 | 13120 |
| 38 | 620 | 3 × Lantus AVE0010 | 4.5 | 41684 |
| 39 | 657 | Lantus form., AVE0010, | 4 | 9309 |
| | | polysorbate 80, | | |
| | | methionine | | |
| 40 | 658 | Lantus form., AVE0010, | 4 | 767 |
| | | poloxamer 188, | | |
| | | methionine | | |
| 41 | 659 | Lantus form., AVE0010, | 4 | 1040 |
| | | benzalkonium chloride, | | |
| | | methionine | | |
| 42 | 660 | Lantus form., AVE0010, | 4.5 | 16803 |
| | | polysorbate 80, | | |
| | | methionine | | |
| 43 | 661 | Lantus form., AVE0010, | 4.5 | 689 |
| | | poloxame 188, | | |
| | | methionine | | |
| 44 | 662 | Lantus form., AVE0010, | 4.5 | 942 |
| | | benzalkonium chloride, | | |
| | | methionine | | |

### 5. THT Test

Methionine has no influence on the tendency to form fibrils. The formulations

| | | Fluorescence intensity |
|---|---|---|
| No. | Composition | at 486 nm |
| 2 | AVE0010 standard | 518 |
| 4 | Lantus standard | 1566 |
| 8 | Lantus form., AVE0010, methionine, | 2053 |
| | pH 4 | |
| 25 | Lantus form., AVE0010, methionine, | 5429 |
| | pH 4.5 | |

have fluorescence values like the reference formulations (no. 2 and 4). With values below approximately 6000, no tendency to form fibrils is present.

When AVE0010, Lantus, and methionine are combined with acetate buffer with or without polysorbate 20 at pH 4, there is a greater tendency to form fibrils:

| | | Fluorescence |
|---|---|---|
| No. | Composition | intensity at 486 nm |
| 2 | AVE0010 standard | 518 |
| 4 | Lantus standard | 1566 |
| 9 | Lantus form., AVE0010, ½ acetate, Met, pH 4 | 18814 |
| 13 | Lantus form., AVE0010, polysorbate 20, ½ | 22200 |
| | acetate, Met, pH 4 | |
| 14 | Lantus form, AVE0010, polysorbate 20, acetate, | 134093 |
| | Met, pH 4 | |

The values for formulations 13 and 14 lie clearly above the threshold for a tendency to form fibrils.

### 6.1 Summary

Polysorbate 20 and polysorbate 80 can lead to turbidity, which is detectable in the double refraction test. Hence, both of these substances can lead to physical instability of a formulation of AVE0010 and insulin.

The addition of methionine does not lead to physical instability.

### 7. Chemical Stability

### 7.1 Stability at Time Point To

The formulations which comprise methionine (with and without sodium acetate) have the lowest amounts of impurities (overall, approximately 1.2 to 1.5%). The following formulations have low amounts of impurities:
8 Lantus form., AVE0010, methionine, pH 4
9 Lantus form., AVE0010, ½ acetate buffer, methionine, pH 4
11 Lantus form., AVE0010, polysorbate 20, methionine, pH 4
13 Lantus form., AVE0010, ½ acetate buffer, polysorbate 20, methionine, pH 4
14 Lantus form., AVE0010, acetate buffer, polysorbate 20, methionine, pH 4
25 Lantus form., AVE0010, methionine, pH 4.5
26 Lantus form., AVE0010, ½ acetate buffer, methionine, pH 4.5
28 Lantus form., AVE0010, polysorbate 20, methionine, pH 4.5
30 Lantus form., AVE0010, ½ acetate buffer, polysorbate 20, methionine, pH 4.5
31 Lantus form., AVE0010, acetate buffer, polysorbate 20, methionine, pH 4.5

Formulations which did not comprise methionine showed a higher fraction of impurities.

Polysorbate 20 has no negative influence on the chemical stability of the formulations.

Acetate buffer has no negative influence on chemical stability when it is combined with methionine and polysorbate 20.

When lysine is present in the formulations, the sum of impurities is greater. The same is true for formulations which comprise polysorbate 80, poloxamer 188, and benzalkonium chloride.

Determining the impurities of insulin glargine revealed that all formulations had comparable amounts of impurities (0.3 to 0.4%).

7.2 Stability after 1 month

### 7.2.1 Impurities of AVE0010

The content of oxidized methionine in the formulations was analyzed. The sequence of AVE0010 has one methionine residue at position 14. The sequence of insulin glargine has no methionine residues. Therefore, the content of oxidized methionine is indicative of oxidation of AVE0010 at the methionine residue. The data are summarized in FIG. 1. Overall, the data show that, without methionine at a pH of 4.5, the fraction of Met(ox) is higher than at pH 4.0. Without methionine as a constituent of the formulations, the fractions of Met(ox) are greatest when the content of insulin glargine is increased or the content of AVE0010 is reduced.

Generally, the greatest fractions of Met(ox) were measured after storage at 40° C./75% relative humidity. Here, the lowest fractions of Met(ox)-AVE0010 (<1%) are to be found in the formulations 8, 9, 11, 13, 14, 25, 26, 28, 30, and 31. The values of these formulations are in the range of the values for the AVE0010 reference formulations no. 1 and 2 (frame in FIG. 1).

The impurities of AVE0010 after 1 month without Met (ox) are represented in FIG. 2. The frames show the values of the AVE0010 reference formulations at 25° C. and at 40° C. Formulations which have the same or better impurity values than the AVE0010 reference formulations are within or below the frames. This is true for the formulations 24, 25, 26, 28, 29, 30, 31, 33, and 34 (40° C.). Impurity values which are above the impurity values of the AVE0010 reference formulations indicate impurities of insulin glargine. Generally, formulations having a pH of 4.5 have fewer impurities than at a pH of 4.0.

The following formulations have, after storage for one month at 40° C., the lowest content of Met(ox) and, simultaneously, the lowest content of other impurities (comparison of FIGS. 1 and 2). They are better than or the same as the AVE0010 reference formulations:
25 Lantus form., AVE0010, methionine, pH 4.5
26 Lantus form., AVE0010, ½ acetate buffer, methionine, pH 4.5
28 Lantus form., AVE0010, polysorbate 20, methionine, pH 4.5
30 Lantus form., AVE0010, ½ acetate buffer, polysorbate 20, methionine, pH 4.5

These formulations also belonged to those formulations which have at time point to the lowest amounts of AVE0010 impurities. All formulations comprise methionine. Polysorbate 20 has no negative effects on the impurities.

The impurities of insulin glargine are represented in FIG. 3. Formulations 3 and 4 are the reference formulations for insulin glargine. The values of these formulations are indicated as narrow frames. All formulations which were identified with regard to AVE0010 impurities as the best formulations (broad frames, more particularly formulations 25, 26, 28, and 30) are, with regard to insulin glargine impurities, better than the insulin glargine reference formulations (approximately 1.5 to 2.4% at 40° C.).

Hence, it can be deduced from this experiment that methionine engenders an increased storage stability of a composition comprising an insulin (e.g., Lantus) and a GLP-1 agonist (e.g., AVE0010). The addition of methionine engenders chemical integrity of this composition.

### 8. Conclusions

The data described herein lead to the following conclusions:
Methionine leads to an increased chemical stability and has no negative effects on the physical stability of formulations of a combination of a GLP-1 agonist, more particularly AVE0010, and an insulin, more particularly Lantus. Therefore, methionine is advantageous as a constituent of these compositions.
Acetate can lead to physical instability. This instability is greater with increasing acetate concentration. Therefore, formulations of a combination of a GLP-1 agonist, more particularly AVE0010, and an insulin, more particularly Lantus, which are free of acetate are advantageous compared with corresponding compositions which comprise acetate.
Polysorbate 20 has no negative influence on the physical and the chemical stability of formulations of a combination of a GLP-1 agonist, more particularly AVE0010, and an insulin, more particularly Lantus. By combining acetate at lower concentrations (½ acetate) with polysorbate 20, the negative effects of acetate can be partially compensated. In acetate-free compositions, the addition of polysorbate 20 does not lead to any advantages. Therefore, formulations of a combination of a GLP-1 agonist, more particularly AVE0010, and an insulin, more particularly Lantus, should be prepared which are free of polysorbate 20.
Lysine (at normal and higher concentrations), benzalkonium chloride, polysorbate 80, and poloxamer 188 already showed chemical instability at the beginning of the studies (t0). For lysine, this is also true for the results of the THT test.

### EXAMPLE 2

The “3 pens cover all” concept (FIG. 4)
3 premix pens having 3 different predetermined proportions:
   (a) Mix A: 100 U Lantus+66.66 μg AVE0010 per mL
   (b) Mix B: 100 U Lantus+40 μg AVE0010 per mL
   (c) Mix C: 100 U Lantus+25 μg AVE0010 per mL
Use of the 3 premix pens: The exemplary table in FIG. 4 proceeds from a therapeutic range of 15 to 80 U per dose of Lantus and 10 to 20 μg AVE0010. For a particular patient, a dose of Lantus to be administered is set or predetermined. The predetermined dose is looked up in the left-hand column. When a corresponding AVE0010 dose in the range from 10 to 20 μg is mentioned in the columns MIX A-MIX C, the corresponding MIX is selected, metered, and administered. The ranges are overlapping: for example, when 26 to 30 U Lantus is required, Mix A or MIX B (having a higher dose of AVE0010) could be selected. Accordingly, this is true for MIX B and C. If, for example, a dose of 50 U of insulin is determined, then 0.5 ml of MIX B or MIX C is to be metered. This dose contains 20 μg (MIX B) or 12.5 μg (MIX C) of AVE0010.
Conclusion: Assuming that a probable AVE0010 effect in the range from 10 to 15 μg and a therapeutic effect in the range from 15 to 22 μg is achieved, almost all patients who take Lantus doses of 15-80 U can likewise receive therapeutic doses of AVE0010 in the range from 10 to 20 μg when they use one of the three premix pens, which contain three different Lantus: AVE0010 ratios (Mix A, B, or C). Due to the broad range of possible ratios of Lantus to AVE0010, the ratios in the pens can be fine-tuned such that a desired dose of AVE0010 is included for every dose of Lantus in at least one pen.

Sanofi-Aventis Deutschland GmbH DE2009/205 Dr. FI/Ba

1-27. (canceled)

28. An injectable composition comprising a glucagon-like peptide 1 (GLP-1) agonist and/or a pharmaceutically acceptable salt thereof, an insulin, methionine, sodium hydroxide and/or hydrochloric acid in a quantity sufficient to achieve a pH in the range of about 4 to about 5, and water.

29. The injectable composition of claim 28, wherein the composition comprises sodium hydroxide and/or hydrochloric acid in a quantity sufficient to achieve a pH of 4.5.

30. The injectable composition of claim 28, wherein the composition comprises methionine in an amount ranging from 1 mg/mL to 5 mg/mL.

31. The injectable composition of claim 28, wherein the insulin is selected from the group consisting of Gly (A21)-Arg(B31)-Arg(B32) human insulin, Lys^{B28}Pro^{B29} human insulin, B28Asp human insulin, and B29Lys(ε-tetradecanoyl), desB30 human insulin.

32. The injectable composition of claim 28, wherein the GLP-1 agonist is selected from the group consisting of desPro³⁶exendin-4(1-39)-Lys₆-NH₂ and exendin-4.

33. The injectable composition of claim 28, wherein the composition further comprises a pharmaceutically acceptable preservative and a pharmaceutically acceptable isotonicity agent.

34. The injectable composition of claim 28, wherein the composition further comprises m-cresol and glycerol.

35. The injectable composition of claim 28, wherein the composition further comprises a source of zinc ions.

36. An injectable composition comprising:
(a) desPro³⁶exendin-4 (1-39)-Lys₆-NH₂,
(b) Gly(A21)-Arg(B31)-Arg(B32) human insulin,
(c) L-methionine,
(d) m-cresol,
(e) glycerol,
(f) zinc chloride,
(g) hydrochloric acid or sodium hydroxide in a quantity sufficient to achieve a pH of about 4.5,
and
(h) water.

37. The injectable composition of claim 36, wherein the composition exhibits at least 90% activity after being stored for at least 2 months.

38. The injectable composition of claim 37, wherein the composition exhibits at least about 90% activity after being stored for at least about 4 months.

39. The injectable composition of claim 38, wherein the composition exhibits at least about 95% activity after being stored for at least about 4 months.

40. The injectable composition of claim 39, wherein the composition exhibits at least about 90% activity after being stored for at least about 6 months.

41. The injectable composition of claim 40, wherein the composition exhibits at least about 95% activity after being stored for at least about 6 months.

42. The injectable composition of claim 41, wherein the composition exhibits at least about 95% activity after being stored for at least about 6 months at a temperature greater than 25° C.

43. A method for treating a subject, wherein the treatment comprises administering the injectable composition of claim 36 to the subject.

44. The method of claim 43, wherein the injectable composition is used in a combination therapy with metformin.

45. The method of claim 44, wherein the subject is afflicted with diabetes mellitus.

46. The method of claim 45, wherein the subject is afflicted with type II diabetes mellitus.


SEQUENCE LISTING (4 sequences)

SEQ ID NO: 1 - protein, 44 aa, synthetic construct
  REGION 1..44: note = AVE0010
HGEGTFTSDLSKQMEEEAVRLFIEWLKNGGPSSGAPPSKKKKKK

SEQ ID NO: 2 - protein, 39 aa, synthetic construct
  REGION 1..39: note = Exendin-4
HGEGTFTSDLSKQMEEEAVRLFIEWLKNGGPSSGAPPPS

SEQ ID NO: 3 - protein, 39 aa, synthetic construct
  REGION 1..39: note = Exendin-3
HSDGTFTSDLSKQMEEEAVRLFIEWLKNGGPSSGAPPPS

SEQ ID NO: 4 - protein, 30 aa, synthetic construct
  REGION 1..30: note = GLP-1
HAEGTFTSDVSSYLEGQAAKEFIAWLVKGR